(19) United States
(12) Patent Application Publication
(10) Pub. No.: US 2007/0086977 A1
(43) Pub. Date: Apr. 19, 2007

(54) Method of treating dermatological conditions

(76) Inventor: Richard F. Stockel, Bridgewater, NJ (US)

Correspondence Address: Law Offices of Jack Matalon, 32 Shelley Rd., Springfield, NJ, 07081-2529, US

(21) Appl. No.: 11/637,450

(22) Filed: Dec. 12, 2006

Related U.S. Application Data
(63) Continuation-in-part of application No. 10/741,346, filed on Dec. 22, 2003, now abandoned.

(51) Int. Cl. A61K 31/785; A61K 31/155

(52) U.S. Cl. 424/78.27; 514/635

(57) ABSTRACT

A method of treating a patient having a dermatological condition which comprises administering to the patient a composition comprising a complex characterized as having a maximum water solubility of about 5 wt. % and further characterized as being a complex that is formed by a metathesis reaction between a bioactive monomeric or polymeric cationic molecule with a bioactive monomeric or polymeric anionic molecule or by an acid-base reaction between a bioactive monomeric or polymeric free base and a bioactive monomeric or polymeric acid capable of protonating the free base.

### REFERENCE TO RELATED APPLICATIONS

This application is a continuation-in-part of application Ser. No. 10/741,346 filed Dec. 22, 2003, the disclosure of which is incorporated herein in its entirety.

### FIELD OF THE INVENTION

The invention relates to the treatment of skin disorders with a broad range of compounds having exceptional antimicrobial, antibiotic, antibacterial and/or antifungal activity and reduced undesirable side effects such as skin irritation. The unique bioactive complexes employed in the invention have been found to be extremely useful for the treatment of many skin condition such as, but not limited to, ichthyosis, eczema, dry skin psoriasis, pruritus, palmar, plantar hyperkeratosis, acne, keratoses, herpes virus, and the like.

The invention provides compositions and methods for the alleviation of both visible and non-visible, i.e., pre-emergent, dermatological lesions associated with changes in normal keratinization, cutaneous infection, epidermal formation or pilosebaceous function such as acne, psoriasis, seborrhea, ingrown hairs and pseudofolliculitis barbae, and hyper-pigmented skin.

The present invention significantly expands the options for treatment of dermatological conditions by allowing the medical professional to choose from a plethora of bioactive agents having the proper chemical characteristics required to prepare a composition to be used for a specific skin condition. For example a cationic (or conjugate base) molecule with antimicrobial, antibacterial, antifungal or antibiotic properties can be combined with a selected anionic (or conjugate acid, respectively) molecule to provide the desired therapeutic outcome.

### BACKGROUND OF THE INVENTION

The prior art is replete with various approaches to the treatment of dermatological conditions. Benzoyl peroxide is well known as a medicament for the treatment of acne. Often the benzoyl peroxide is combined with an antibacterial agent or an antibiotic to extend its spectrum of activity as disclosed in, e.g., U.S. Pat. No. 5,767,098. However there are several disadvantages to this combination approach. With prolonged usage that is typically required for the treatment of acne, the bacterial flora become resistant thus rendering the antibacterial agent or the antibiotic less effective in subsequent treatment. Moreover, the benzoyl peroxide component of the combination is oxidatively unstable.

Numerous publications and patents disclose the use of α-hydroxy acids for the treatment of dermatological conditions. For example, U.S. Pat. No. 4,363,815 discloses the use of such compounds for the treatment of dry skin, ichthyosis, plantar hyperkeratosis, Darier's disease, keratoses, acne, psoriasis, eczema, pruritus, warts and herpes virus. Other patents describe the use of various incipients to lessen skin irritation and stinging, e.g., lactate salts, amphoteric salts (see U.S. Pat. No. 5,420,106), ascorbic acid derivatives (U.S. Pat. No. 5,703,122), amino salts (see Cosmetics and Toiletries, volume 113, March 1998, p. 55).

Salicyclic acid is frequently disclosed as an active ingredient for the treatment of a wide variety of skin conditions, e.g., psoriasis, skin atrophy, skin wrinkles, acne, etc., see U.S. Pat. Nos. 5,776,920; 5,780,457; 5,780,458; 6,436,417. In all of these cases, salicyclic acid must be modified with other incipients to prevent undesirable side reactions such as skin irritation and the like.

There is a need for a safe and efficacious course of treatment for severe acne. The only treatment to date which has proven to be uniformly effective is isotretinoin which is orally administered. This medication has many undesirable side effects including the possibility of causing birth defects when administered to pregnant women.

### DETAILS OF THE INVENTION

The invention relates to the treatment of dermatological conditions by administering to the patient that is suffering from such conditions a composition comprising topical bioactive complexes that have antimicrobial, antibiotic, antibacterial and/or antifungal properties.

The bioactive complex employed in the invention is characterized as having a maximum water solubility of about 5 wt. %, preferably a maximum water solubility of 2 wt. %. The bioactive complex is further characterized as being a complex that is formed by a metathesis reaction between a bioactive monomeric or polymeric cationic molecule with a bioactive monomeric or polymeric anionic molecule or by an acid-base reaction between a bioactive monomeric or polymeric free base and a bioactive monomeric or polymeric acid capable of protonating the free base. The bioactive complexes have been found to be extremely effective against a wide variety of microorganisms, e.g., bacteria and fungi. Moreover, the complexes have important safety, efficacy and toxicity implications since the bioactive components employed in the preparation of the complexes by either a metathesis or an acid-base reaction are typically those that have been approved for use by the EPA or the FDA.

The bioactive complexes tend to have low water solubility and therefore may have prolonged extended-release properties. For many, but most certainly not all, applications, it is desirable to apply the complexes topically m the form of emulsions, nano-emulsions, microemulsions, gels, dispersions or creams. When a particular skin condition so dictates, the complex may be orally administered in the form of tablets or capsules by compounding the complexes with the usual excipients as is well known in the pharmaceutical field.

The dosage of the bioactive complexes to be administered to patients having dermatological conditions will, of course, depend on the particular dermatological condition and its severity. Since the bioactive components of the complexes are themselves well known in the art, the skilled medical practitioner will have no problem in devising a dosage regimen for each particular patient.

### The Complexes

As mentioned above, the bioactive complexes employed in the method of the invention are characterized as having a maximum water solubility of about 5 wt. %, preferably 2 wt. %, and are further characterized as having been formed as the result of a metathesis reaction between a bioactive monomeric or polymeric cationic molecule with a bioactive monomeric or polymeric anionic molecule or by an acid-base reaction between a bioactive monomeric or polymeric free base and a bioactive monomeric or polymeric acid capable of protonating the free base.

In respect to the metathesis reaction, the cationic molecule may be an amidine; a guanidine biguanide; a quaternary amine; an amine acid salt of an azole, an amine acid salt of an antibiotic; a gemini quaternary amine; a dendrimeric quaternary amine; or an aminosaccharide salt. Preferably, the cationic molecule is monomeric in nature and comprises an amine acid salt of an azole or an amine acid salt of an antibiotic. Suitable azoles include cloconazole, clotrimazole, cyproconazole, fenbucanozole, fiucytosine, miconazole, myclobutanil, propiconazole, tebuconazole andtriadimefon. Suitable antibiotics include clinafloxacin, clindamycin, doxycycline, erythromycin, lincomycin, minocycline, tazarotene and tetracycline.

The anionic molecule may be carboxylic; hydroxy carboxylic; β-keto carboxylic; phenolic; or sulfonamide. Suitable carboxylics include adapalene, azelaic, isotretinoin, pantothenic, retinoic; tretinoin and undecylenic. Suitable hydroxy carboxylics include gluconic, glycolic, glyceric, lactic and salicyclic. The phenolic may be, e.g., hexylresorcinol or thymol.

In respect to the acid-base reaction, the preferred bases are azoles and antibiotics. Particularly preferred azoles and antibiotic are those recited above. The preferred acid may be carboxylic; hydroxy carboxylic; β-keto carboxylic; phenolic; or sulfonamide, and the preferred carboxylics, hydroxy carboxylics and phenols are those recited above.

### The Metathesis Reaction

As noted in the McGraw-Hill Dictionary of Scientific and Technical Terms (5^{th} Edition, 1994), metathesis is a reaction involving the exchange of elements or groups as in the general reaction:
AX+BY→AY+BX.

The metathesis reaction is straight forward and can be readily carried out in aqueous solutions using water alone or a mixture of water and up to about 85 wt. % of a solvent such as a C₁-C₄ alcohol, e.g., methanol, ethanol, isopropanol, n-butanol, etc. Typically the water alone or water-alcohol solvent will be utilized in an amount of about 40 to about 85 wt. %, based on the weight of the reaction mixture.

An alkali or alkaline earth metal (e.g., Na, K, Li, Ca, etc.) salt of the selected bioactive anionic monomer or polymer is formed by reacting it with an equivalent amount of an alkali or alkaline earth metal hydroxide in water or water-alcohol solution. An acid salt, e.g., acetate, hydrohalide, gluconate, sulfate, etc. of the selected free base bioactive monomer or polymer is formed by reacting it with an equivalent amount of an acid such as acetic, hydrochloric, hydrobromic, gluconic acid, sulfuric, etc. in water or water-alcohol solution.

Thereafter, an equivalent amount of the aqueous alkali or alkaline earth metal salt solution of the selected bioactive anionic monomer or polymer is mixed with the aqueous acid salt solution of the selected cationic monomer or polymer. The concentration of the reactants can vary from about 20 wt. % to about 60 wt. % of the total reaction mixture. Mixing is continued at room temperature for several minutes up to about one hour. The reaction product may be readily recovered by decantation of the supernatant layer (which contains the byproduct salts) or by filtration. The solid layer consisting of the complex may be used as is for many of the materials recited above or dried (e.g., in air, in vacuuo at a temperature of about 50 to about 130° C., etc.). If desired, the complex may be recrystallized using a solvent such that the solubility of the complex in the solvent is low at room temperature, but the solubility increases significantly near the boiling point of the solvent.

### The Acid-Base Reaction

It is preferred to use an acid-base reaction to prepare the desired complex if the selected bioactive monomeric or polymeric acid is capable of protonating the selected monomeric or polymeric free base. The use of the acid-base reaction avoids the necessity of forming an alkali metal salt of the selected bioactive anionic monomer or polymer and the acid salt of the selected bioactive cationic monomer or polymer and having to dispose of the salt byproduct.

The acid-base reaction of a conjugate base (i.e., the free base) of the selected bioactive cationic monomer or polymer with the conjugate acid (protonated) of the selected bioactive anionic monomer or polymer may be illustrated by the following example:

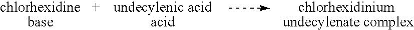

In order for the acid-base reaction to proceed, the acid component must have a transferable proton (Pₖₐ) to a basic (P_{kb}) molecule. The acid-base reaction is usually conducted in refluxing alcohol (e.g., a C₁-C₄ alcohol such as methanol, ethanol, isopropanol, n-butanol, etc.) or aqueous alcoholic solution (e.g., about 10 to about 90 wt. % water) and the reaction is typically complete in one hour or less. The complex may be readily recovered from the reaction mixture by filtration, air drying, removal of the solvent in vacuuo at a temperature of about 50 to about 130° C., etc. If desired, the complex may be recrystallized using a solvent such that the solubility of the complex in the solvent is low at room temperature, but the solubility increases significantly near the boiling point of the solvent.

The acid-base reaction is particularly advantageous for the formation of bioactive azole complexes of bioactive components that have a protonic hydrogen capable of transfer to a base nitrogen in an azole molecule. The azoles are either imidazole or triazole derivatives. If the azole can be protonated, then it can be subsequently reacted with an anionic monomeric or polymeric bioactive component.

### Formation of Emulsions of the Complexes

As mentioned above, the complexes employed in the invention have limited water solubility. Therefore, for many dermatological medicaments it is desirable to utilize the complexes in the form of emulsions, nanoemulsions or microemulsions. The following is a generalized procedure for preparing emulsions, nanoemulsions or microemulsions of the complexes.

First, the complex is dissolved in the minimum amount of a solvent that will completely dissolve the selected complex in the amount that is intended for use in the desired dermatological medicament. The solvent of choice will be one with the appropriate Hildebrand solubility parameter. The solubility parameter is a numerical value that indicates the relative solvency behavior of a specific solvent. Hildebrand solubility parameters of about 8.5 to about 22.0 are generally suitable for solubilization of the complexes. Exemplary solvents with the requisite Hildebrand solubility parameters include ethanol, glycerine, propylene glycol, sorbitol, methanol and the like.

The desirable Hildebrand solubility parameter will depend on the ionic/covalent bonding energies of the complexes. The correct solvent will be one having a relatively low Hildebrand solubility parameter if the bonding has more covalency and a relatively high Hildebrand solubility parameter if the bonding is more ionic. Of course, combinations of correct solvents may also be utilized to dissolve the complexes.

Thereafter, a surfactant is added to the dissolved complex. The surfactant may be cationic, anionic or amphoteric in nature, and combinations of the different types or combinations of the same type of surfactants may be use. Preferably, the surfactant will be amphoteric or nonionic in nature. Highly negative anionic surfactants are not very functional.

The last step is to dilute the complex-solvent-surfactant composition with water to the concentration desired for the selected dermatological medicament so as to form an emulsion, nanoemulsion or microemulsion depending on the micellar size and the choice of solvents/cosolvents.

### The Surfactants

For the purposes of this invention, it is preferred that the surfactants employed in the formation of emulsions, nanoemulsions or microemulsions of the complexes are generally of the nonionic or amphoteric type or combinations of one or more nonionics, one or more amphoterics or one or more nonionics in combination with one or more amphoterics. Also, a cationic-amphoteric or cationic-nonionic surfactant system can be utilized to provide satisfactory results. Highly charged anionic surfactants are less desirable since they have the potential to reduce the bioactivity of the complexes by causing some degree of precipitation, thereby lessening the effectiveness of the complexes.

It has also been found that cationic phospholipids, preferably in combination with nonionic and/or amphoteric surfactants are effective in the formation of microemulsions or emulsions of the complexes.

Surfactants that carry a positive charge in strongly acidic media carry a negative charge in strongly basic media, and form zwitterionic species at intermediate pH levels are amphoteric. The preferred pH range for stability and effectiveness is about 5.0 to about 9.0. Within this pH range, the amphoteric surfactant is mostly or fully in the zwitter (neutral) form, thereby negating any dilution of bioactivity of the complexes, provided that the surfactant is employed in the preferred concentration range of about 0.25 to about 6.0 wt. %, based on the weight of the complex in the final formulation.

The following surfactants have been found to be effective in the formation of emulsions, nanoemulsions and microemulsions of the complexes: amphoteric amidobetaines; nonionic polyethoxylated sorbital esters, polycondensates of ethylene oxide-propylene oxides (polyoxamers), polyethoxylated hydrogenated castor oils, and certain cationic phospholipids.

Suitable examples of amidobetaines include cocoamidoethyl betaine, cocoamido-propyl betaine; and mixtures thereof. Other suitable amphoteric surfactants include long chain imidazole derivatives such as the product marketed under the trade name “Miranol C2M” by Rhodia and long chain betaines such as the product marketed under the trade name “Empigen BB” by Huntsman Corporation, and mixtures thereof.

Suitable nonionic surfactants include polyethoxylated sorbitol esters, especially poly-ethoxylated sorbital monoesters, e.g., PEG sorbitan di-isostearate, and the products marketed under the trade name “Tween” by ICI; polycondensates of ethylene oxide and propylene oxide (polyoxamers), e.g., the products marketed under the trade name “Pluronic” by BASF; condensates of propylene glycol; polyethoxylated hydrogenated castor oil such as the products marketed under the trade name “Cremophors” by BASF; and sorbitan fatty esters marketed by ICI. Other effective nonionic surfactants include the polyalkyl (C₈-C₁₈) glucosides.

Suitable cationic surfactants include D,L-pyrrolidone-5-carboxylic acid salt of ethyl-cocoyl-L-arginate (CAE) marketed by Ajinomoto, and cocoamidopropyl (PTC), lauramidopropyl PG diammonium chloride phosphates and the like marketed by Uniqema. CAE and PTC have significant bioactivity and they therefore can be used as the cation of the binary cationic-anionic bioactive complexes.

The choice of an effective surfactant system will differ somewhat for each bioactive complex. The choice will depend on the surfactant(s)' hydrophilic-lipophilic balance (HLB) to form a stable small particle micelle in an aqueous or aqueous-cosolvent medium.

Other adjuvants useful in formulating the bioactive complexes in o/w or w/o type creams, gels, lotions and the like include: polyether-modified silicone, cyclic silicone, methyl polysilicone, polyoxyethylene castor oil, cetostearyl alcohol, neopentyl glycol dicaprate, sorbitan monostearate, polyvinyl alcohol, glycerin, “Carbox”, glyceryl ether, cholesteryl isostearate, ethanol, isopropanol, glycerol monostearate PEG 100 stearate, hydroxymethyl cellulose, cetyl alcohol, lauryl glucoside and the like.

The following examples shall serve to illustrate the various embodiments of the invention. The examples are given solely for the purpose of illustration and are not to be construed as limitations of the present invention since many variations are possible without departing from the spirit and scope of the invention. Unless otherwise indicated, all parts and percentages are on a weight basis.

### EXAMPLE 1

This example pertains to a lotion formulation containing a chlorhexidine-salicylate complex.

| Ingredient | Weight % |
|---|---|
| Complex | 0.5 |
| “Tego Betaine ZF” | 1.5 |
| Propylene Glycol | 15.0 |
| Propylene Glycol Dicaprylate-Dicaprate | 8.0 |
| Hydroxyethyl Cellulose | 0.25 |
| PEG 40 Stearate | 2.50 |
| “Stearath-2” | 1.00 |
| Water | QS |

### EXAMPLE 2

This example pertains to a cream formulation containing a miconazole-azelaic complex.

| Ingredient | Weight % |
|---|---|
| Complex | 0.75 |
| Propylene Glycol | 15.00 |
| “Steareth-20” | 1.50 |
| Hydroxyethyl Cellulose | 0.50 |
| Glyceryl Monostearate | 0.15 |
| Propylene Glycol Monolaurate | 2.00 |
| “Tween-20” | 5.00 |
| Water | QS |

### EXAMPLE 3

This example pertains to an aqueous microemulsion containing a complex resulting from the reaction of N-lauroyl-L-arginine-ethyl ester hydrochloride salt with sodium lactate.

| Ingredient | Weight % |
|---|---|
| Complex | 1.00 |
| Ethanol | 55.00 |
| Hydroxypropyl Cellulose | 0.50 |
| “Tego Betaine ZF” | 2.00 |
| Propylene Glycol | 10.00 |
| Dye | 0.020 |
| Water | QS |

What is claimed is:

1. A method of treating a patient having a dermatological condition which comprises administering to the patient a composition comprising a complex characterized as having a maximum water solubility of about 5 wt. % and further characterized as being a complex that is formed by a metathesis reaction between a bioactive monomeric or polymeric cationic molecule with a bioactive monomeric or polymeric anionic molecule or by an acid-base reaction between a bioactive monomeric or polymeric free base and a bioactive monomeric or polymeric acid capable of protonating the free base.

2. The method of claim 1 wherein said complex has a maximum water solubility of 2 wt. %.

3. The method of claim 1 wherein the cationic molecule is selected from the group consisting of an amidine; a guanidine biguanide; a quaternary amine; an amine acid salt of an azole, an amine acid salt of an antibiotic; a gemini quaternary amine; a dendrimeric quaternary amine; and an aminosaccharide salt.

4. The method of claim 3 wherein the amidine is selected from the group consisting of propamidine and dibrompropramidine.

5. The method of claim 3 wherein the biguanide is selected from the group consisting of alexidine, hexetidine and a chlorhexidine salt.

6. The method of claim 3 wherein the gemini quaternary amine comprises a ethandiyl-α, ω-bis(dodecyldimethyl)ammonium halide.

7. The method of claim 3 wherein the quaternary amine is selected from the group consisting of benzalkonium chloride, cetyl pyridinium chloride and didecyldimethyl ammonium chloride

8. The method of claim 3 wherein the complex is selected from the group consisting of a polybiguanide, a polyguanidine, a polyionene, a polyaminosaccharide and a quaternary ammonium dendrimeric biocide.

9. The method of claim 8 wherein the polybiguanide comprises a polyhexamethylene biguanide HCl salt.

10. The method of claim 8 wherein the polyguanidine comprises a polyhexamethylene guanidine HCl salt.

11. The method of claim 8 wherein the polyionene comprises poly[oxyethylene (dimethylimino)ethylene(dimethylimino)ethylene] dichloride.

12. The method of claim 8 wherein the polyaminosaccharide comprises a chitosan salt.

13. The method of claim 3 wherein the cationic molecule comprises an amine acid salt of an azole.

14. The method of claim 13 wherein the azole is selected from the group consisting of cloconazole, clotrimazole, cyproconazole, fenbucanozole, fiucytosine, miconazole, myclobutanil, propiconazole, tebuconazole and triadimefon.

15. The method of claim 3 wherein the cationic molecule comprises an amine acid salt of an antibiotic.

16. The method as defined in claim 15 wherein the antibiotic is selected from the group consisting of clinafloxacin, clindamycin, doxycycline, erythromycin, lincomycin, minocycline, tazarotene and tetracycline.

17. The method of claim 1 wherein the anionic molecule is selected from the group consisting of carboxylic; hydroxy carboxylic; β-keto carboxylic; phenolic; and sulfonamide.

18. The method of claim 17 wherein the carboxylic is selected from the group consisting of adapalene, azelaic, isotretinoin, pantothenic, retinoic, tretinoin and undecylenic.

19. The method of claim 17 wherein the hydroxy carboxylic is selected form the group consisting of gluconic, glycolic, glyceric, lactic and salicyclic.

20. The method of claim 17 wherein the phenolic is selected from the group consisting of hexylresorcinol and thymol.

21. The method of claim 1 wherein the base comprises an azole.

22. The method of claim 21 wherein the azole is selected from the group consisting of cloconazole, clotrimazole, cyproconazole, fenbucanozole, fiucytosine, miconazole, myclobutanil, propiconazole, tebuconazole and triadimefon.

23. The method of claim 1 wherein the base comprises an antibiotic.

24. The method of claim 23 wherein the antibiotic is selected from the group consisting of clinafloxacin, clindamycin, doxycycline, erythromycin, lincomycin, minocycline, tazarotene and tetracycline.

25. The method of claim 1 wherein the complex is administered to the patient: (a) topically in the form of an emulsion, nanoemulsion, microemulsion, gel, dispersion or cream or (b) orally in the form of a tablet or a capsule.
